(19) United States
(12) Patent Application Publication
(10) Pub. No.: US 2005/0239197 A1
(43) Pub. Date: Oct. 27, 2005

(54) Lid element

(75) Inventors: Andreas Katerkamp, Munster (DE); Uwe Brinkmann, Laer (DE); Frank Grawe, Ochtrup (DE); Goran Key, Osnabruck (DE); Sabine Schreiber, Ascheberg (DE); Jochen Uckelmann, Munster (DE)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: 02-Scan GmbH, Munster (DE)

(21) Appl. No.: 10/503,266

(22) PCT Filed: Jan. 24, 2003
(86) PCT No.: PCT/DE03/00219

§ 371 Date: Aug. 2, 2004

(30) Foreign Application Priority Data
Feb. 1, 2002 (DE) 102 04 531.3

(51) Int. Cl. C12M 1/00

(52) U.S. Cl. 435/292.1

(57) ABSTRACT

The invention relates to a lid element which is placed on cell culture vessels in which cells contained in a liquid medium are accommodated. The lid element according to the invention is intended to allow determination of metabolism activities of cells which are contained in the cell culture vessels, by means of optical measurement methods, which can be carried out with simple handling by means of laboratory personnel. Light-guiding elements are provided on the lid element which can be fitted to the cell culture vessel and, when the lid element is fitted, project into the interior of cavities in the cell vessel. At least one optically sensitive layer is formed on one end surface and/or on the outer envelope surface of the light-guiding elements, which are preferably optical waveguides in the form of rods, for detection of chemical substance concentrations which change within the cavities.

The invention relates to a lid element, which can be placed on cell culture vessels, such as Petri dishes and preferably microtitre plates, and to an apparatus and a method using a lid element such as this for detection of metabolism activity of cells which are contained in liquid media. The invention can advantageously be used, for example, for investigations into the effects of different environmental and (bio) chemical substance influences on the vitality of cells. It is also possible to carry out investigations relating to the improvement of cultivation conditions for the cells in order, for example, to increase the formation rate of biomolecules such as different proteins which are formed by cells.

The expression cells is intended to mean, for example, microorganisms, cells of fungi as well as human, animal and plant cells, for example cell line cells such as HL-60 (human, promyeloblast), U-937 (human, lymphoma) MCF-7 (human, mamacarcinoma), CACO-2 (human, coloncarconoma, J774A1 (murin, macrophage), 3T3 (murin, fibroblast), BHK-12 (hamsters, kidneys), or else primary cells such as those which may be obtained by biopsies or blood.

DE 199 03 506 A1 discloses an appropriate solution in which the change in oxygen concentration within a liquid medium in which cells are contained is measured in specifically designed vessels, and this change is used as a measure of the metabolism activity of the cultivated cells.

The vessels described there have a specific shape, and the sensor membrane to be used is arranged in a defined manner within the vessels, in order to avoid measurement errors. One disadvantage is that the sensor membrane is arranged on the base of the cell culture vessel on which the cells are also located. In particular, this detracts from the cultivation conditions for the cells.

Furthermore, U.S. Pat. No. 5,567,598 discloses an apparatus for verification of microorganisms in liquid samples and for monitoring of the effects of specific chemical substances which influence such microorganisms. According to the teaching provided there, sensor membranes, inter alia, are intended to be arranged at the ends of the wedge-shaped elements, which are referred to there as “prongs”. These wedge-shaped elements are attached to a frame element and are immersed with this sensor membrane in a sample liquid, which is contained in a reservoir. These wedge-shaped elements are, however, partly designed to be hollow in their interior, and are kept closed only on the end face on which the sensor membrane is arranged. The apparatus as described in U.S. Pat. No. 5,567,598 for measurement signal detection from the sensor membrane is highly susceptible to measurement errors since measurements are carried out through the liquid medium and it does not produce quantitative measurement signals, so that this arrangement is not suitable for am automated routine application.

EP 0 425 587 refers to the use of so-called “optodes” for the same area of application. In an example of the solution described there, an optode such as this is intended to be attached to the tip of a probe which can be inserted into a container, with optical waveguides for stimulation and detection device being accommodated within a probe such as this. However, it is worth noting that this solution is intended to be used exclusively in closed systems, which are completely closed off from the environment so that the need of change of substances between the system and the environment is also precluded.

Against this background, the object of the invention is thus to provide a low-cost solution which can be used in a versatile form, by means of which the metabolism activity of cultivated cells can be assessed with a high degree of acceptance by laboratory personnel by means of an optically sensitive layer and optical measurement, taking into account different influencing criteria.

According to the invention, this object is achieved by a lid element which has the features of claim 1, and by an apparatus and a method in which such lid elements are used, as claimed in claim 14 for an apparatus and in claim 21 for a method. Advantageous refinements and developments of the invention can be achieved by the features described in the dependent claims.

The lid elements according to the invention can be fitted in an adaptive form directly to widely differing cell culture vessels which are known per se, and these lid elements allow optical detection and, derived from this, make it possible to determine the metabolism activity of cells which are contained in a liquid medium, for example a nutrition solution. The geometry and dimensions of the lid elements can be adapted relatively easily and can be designed for the normal cell culture vessels which are used in laboratories. For example, a lid element such as this can be designed in a preferred manner for so-called microtitre plates taking account of the respective number and arrangements of the individual cavities (wells).

The lid element according to the invention has at least one light-guiding element, which is preferably an optical waveguide in the form of a rod. These light-guiding elements project into a respective cavity when the lid element is fitted to the respective cell culture vessel.

At least one optically sensitive layer is formed on each of the light-guiding elements. An optically sensitive layer such as this may be formed on the end surface, which projects into the interior of the respective cavity, in/or on an outer envelope surface.

It is, of course, also possible to provide two or more different optically sensitive layers on a light-guiding element such as this.

An optically sensitive layer such as this changes its optical characteristics as a function of the bio) chemical substance concentration which is to be detected and is changed by the metabolism of the cells, in the cavity in the cell culture vessel.

For example, the optical characteristics of optically sensitive layers such as these may change in terms of their luminescence, light transmission or light scatter.

By way of example, it is known for luminescence to be stimulated by suitable light in a layer such as this and that the stimulated luminescence light changes as a function of the substance concentration, and that this change in the luminescence light can be used as a measure of the respective substance concentration.

By way of example, ruthenium complexes are known for determination of the oxygen concentration (Otto S, Wolfbeis (ed.), Fiber Optic Chemical Sensors and Biosensors, Vol. II, CRC Press 1991), which are embedded in a polymer matrix which is permeable to oxygen. These ruthenium complexes have the characteristic that the luminescence intensity changes as a function of the respective oxygen concentration and/or of the oxygen partial pressure. In consequence, the intensity of the luminescence light or the time decay behavior of the luminescence light after a light source which is appropriate to stimulate luminescence is switched off, may be used.

However, since the substances which are suitable for stimulation of luminescence, in particular, and which are embedded in a polymer matrix such as this are subject to a certain amount of aging, and the detection of the luminescence intensity can be corrupted by interference light, it is particularly advantageous to measure the decay time, which changes as a function of the oxygen concentration, of the luminescence by means of a phase shift between the sinusoidal stimulation light and the fluorescence light.

An optically sensitive layer which may be used for a lid element according to the invention may be designed, by way of example, as described in DE 198 31 770 A1.

However, the optically sensitive layers may also be in a different form, without it being possible for luminescence phenomena to occur and to be taken into account.

Thus, for example, an optically sensitive layer may be formed from a substance or may contain such a substance which changes its light transmission characteristics as a function of the respective substance concentration, for example by means of a corresponding successive color shift. In a corresponding manner, more or less light is correspondingly absorbed by an optically sensitive layer such as this, so that the intensity of the transmitted light which passes through such a sensitive layer and strikes an optical detector is likewise a suitable measure. Optical sensor membranes should be mentioned by way of example here, as are known for determination of the carbon dioxide concentration or of the pH value from Otto S, Wolfbeiss (ed.), Fiber Optic Chemical Sensors and Biosensors, Vol. II, CRC Press 1991.

In a further alternative, however, light scatter which occurs with an optically sensitive layer such as this, and which likewise changes as a function of the respective substance concentration, may also be used.

In this case, an optically sensitive layer such as this contains light-scattering particles, in which case these particles may be embedded in a polymer material. This material is influenced by the respective substance concentration and this results in a shift or alignment of the light-scattering or reflecting particles within the layer, so that, in this case as well, the proportion of the light which is transmitted through this layer in the direction of an optical detector is changed as a function of the substance concentration. The layer material in which such particles are embedded may, for example, be in the form of a gel, or in the form of a liquid crystal.

In addition to pure luminescence, light transmission and light scattering measurements, combinations of at least two types of measurement are also possible. Sensible combinations wound be, for example, a luminescence measurement and a light scattering measurement, or a light transmission measurement and a light scattering measurement.

The lid element according to the invention may advantageously have a surface which forms a structure in the area of optical waveguides, which are in the form of rods, as light-guiding elements. In consequence, a structure such as this is formed on that side of the lid element which is opposite such optical waveguides in the form of rods.

By way of example, it is possible for a structure such as this to be in the form of convex projections or concave depressions, in order to make it possible to advantageously influence the light guidance.

Convex projections can thus form plano-convex optical lenses, or concave depressions can form concave lenses, which specifically shape the light to be injected into the optical waveguides, which are in the form of rods. However, the plano-convex lenses can also direct light which emerges on this side of the lid element in a deliberately shaped manner onto an optical detector, or focus it for injection into an optical fiber.

It is also possible to form funnel-shaped depressions, with the light being injected through the respective funnel into the respective optical waveguides, which are in the form of rods. In this case, it is advantageous to form a planar surface within the funnel-shaped area in order to inject and/or output light into and/or out of the respective optical waveguide, which is in the form of a rod.

It is also possible, on their own or in addition to the described structures on the surface of a lid element according to the invention, to additionally deliberately geometrically design the optical waveguides which are provided on the lid element to allow them to have a positive influence on the light guidance within the optical waveguides. In this case, the optical waveguides may have an area which is in the form of a funnel, a truncated cone or a truncated pyramid downwards starting from the top, which area then merges into an area which is in the form of a rod thus resulting in better light guidance characteristics within the optical waveguides for the injection and/or outputting of light.

The optical waveguides, which are entirely in the form of a rod or have only an area which is in the form of a rod, may have a circular, oval, triangular or polygonal cross section, at least in those parts which are in the form of rods.

For example, in this way, two or more optically sensitive layers can be formed relatively easily on the correspondingly planar envelope surface areas on an optical waveguide which is in the form of a rod and has a triangular or polygonal cross section, and it is possible to achieve a considerable degree of isolation between such optically sensitive layers, which are then preferably different.

Particularly for investigations over lengthy time periods, it is advantageous to provide spacers or openings on a lid element according to the invention. These elements avoid there being an hermetic seal between the liquid medium and the environment, so that substance exchange can take place between the environment and the liquid medium. This is particularly important for the aerobic metabolism of cells since, or example, the oxygen which is required can thus enter the liquid medium from the environment, and can reach the cells which consume oxygen, by diffusion.

Spacers such as these may, for example, be projections formed on the lower face, that is to say on the face on which the optical waveguides which are in the form of rods are formed or provided.

However, spacers may also be frame elements which are matched to the normal shape and size of the respectively used cell culture vessels and which can be fitted between the cell culture vessel and the lid element. Spacers of this form which are in the form of frames can also be used to achieve a second effect. It is thus possible to configure a deliberately variable arrangement of the optically sensitive layers within the cavities in a cell culture vessel such as this. For example, the one or else more optically sensitive layers may thus be immersed to a greater or lesser depth in the respective liquid medium or it is even possible for the one or more optically sensitive layer(s) to be arranged above the liquid medium and for the respective measurement of the substance concentration to be carried out there, in the gas area above the liquid.

In the case of lid elements which have openings for gas exchange with the environment, these openings are advantageously closed by gas-permeable membranes so that, for example, it is possible to avoid the undesirable ingress of foreign cells, such as microorganisms.

Reflective or absorbent layers can be formed on the surface of a lid element according to the invention in order to suppress, or at least impede, external and stray light influences, and/or the influence of adjacent cavities. In this case, a reflective or absorbent layer such as this is not formed completely over the surface of a lid element according to the invention, and, instead, the areas for the injection and/or outputting of light into or from the light-guiding elements are, of course, kept free of any such coating.

The lid element according to the invention, various embodiments of which have been described above, can be incorporated in an apparatus for determination of the optical characteristics of the sensitive layers on the light-guiding elements which are influenced by the metabolism of the cells to be cultivated. In this case, light from at least one light source is directed through light-guiding elements (such as optical waveguides which are in the form of rods) provided on the lid element, or is directed through optically sensitive layers that are formed there, and the light which has been influenced by the one or else more optically sensitive layer(s) is measured by means of at least one optical detector, in which case the measurement, as already described above, can be carried out in various ways, for example a luminescence light measurement, a light transmission measurement or a light scatter measurement, or else a combination of at least two of these measurements.

Luminescence measurement devices such as fluorescence scanners/readers and appliances which measure photometrically, for example an ELISA plate reader, can be used for an apparatus such as this, provided that an appropriate optically sensitive layer is formed on the optical waveguides, which are in the form of rods, as light-guiding elements.

However, the light from a light source can also be guided onto or through such optically sensitive layers on the optical waveguides, which are in the form of rods, by means of optical fibers. These optical fibers or further additional optical fibers can also direct the respective light to be measured onto at least one optical detector. If two or more individual cell culture vessels or cell culture vessels with two or more cavities are used, it is advantageous to design the apparatus so as to allow relative movement between the lid element on the cell culture vessel, the light source and the end surfaces of the optical fibers which are used for coupling light into and/or out of the optical waveguides, which are in the form of rods, on the lid element. This allows deliberate positioning with respect to the optically sensitive layer on the respective light-guiding element in the cavity in the cell culture vessel, so that the measurements can be carried out sequentially in the individual cavities. It is, of course, also possible to provide an appropriate relative movement with respect to at least one light source, one optical fiber and/or one optical detector.

In the case of an apparatus such as this, it is advantageous, for the illumination of the optically sensitive layers, to arrange the one or more light sources or the end surfaces of an optical fiber on which the light that is directed onto such optically sensitive layers is output above the lid element, and in consequence also above the openings of the cavities which are formed in the cell culture vessel. Particularly when luminescence stimulation is being used in the optically sensitive layers, the at least one optical detector should also be arranged above the lid element, or at least the end surface of an optical fiber into which the luminescence light is injected or through which the luminescence light is directed at the optical detector, should be arranged appropriately there.

Particularly for the situation where the intensity of light which is directed through an optically sensitive layer is intended to be measured in order to assess the metabolism activity of the cells to be cultivated, it is, however, better to arrange an optical detector appropriately underneath the cell culture vessel or a corresponding end surface of an optical fiber into which this light is injected, and through which the light is directed at an optical detector.

It is also advantageous to carry out a comparison measurement in a cavity which, although it contains a correspondingly identical liquid medium to that in the other cavities, does not contain any metabolism-active cells or additional substances whatsoever, so that this cavity can in consequence be regarded as being normal.

In addition to determination of the oxygen concentration, which has been mentioned a number of times already, the solution according to the invention also makes it possible to determine the CO₂—, H₂—, H⁺—, H₂S—, NH⁴⁺ concentration, and/or the pH value.

Furthermore, it is possible to determine the concentration and/or the change in the concentration of enzyme substrates which have been produced by the metabolism of the cells. In this case, enzyme sensors can be used for optically sensitive layers. However, it is also possible to use enzyme sensors such as these to detect glucose and/or lactate.

The invention will be explained in the following text using examples.

In the figures:

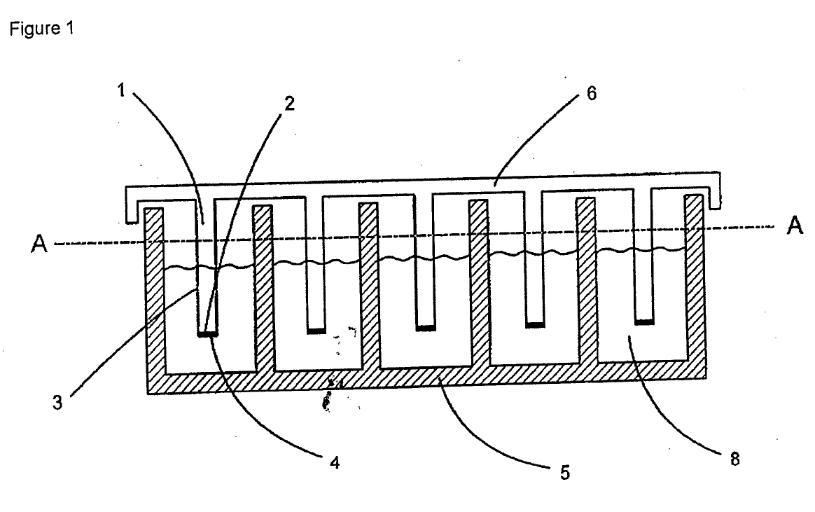

FIG. 1 shows, schematically and in the form of a section, a lid element according to the invention which is placed on a cell culture vessel in the form of a microtitre plate;

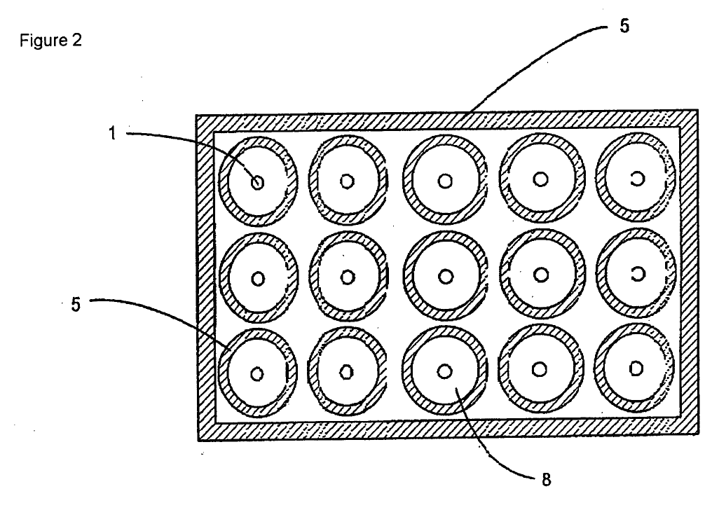

FIG. 2 shows a plan view, in the form of a section, along the line A-A in FIG. 1;

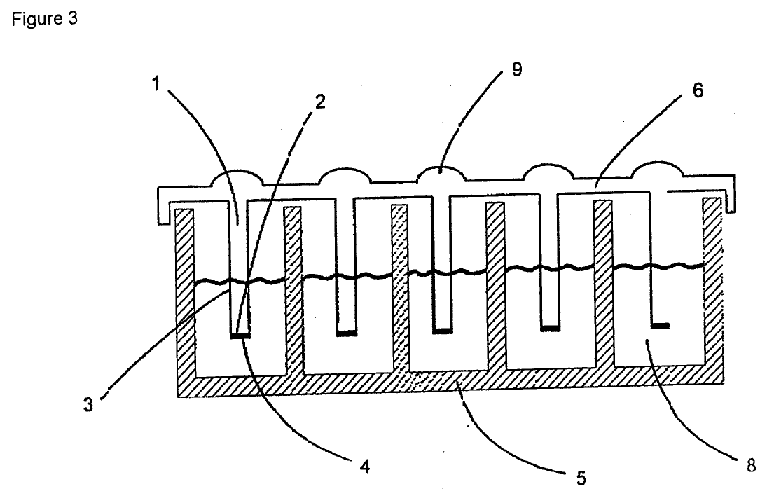

FIG. 3 shows a section illustration of one advantageous development of a lid element according to the invention;

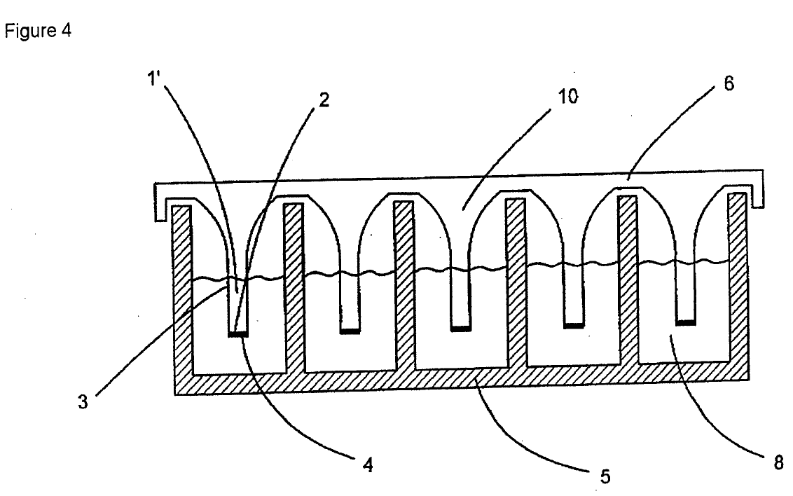

FIG. 4 shows another embodiment of a lid element according to the invention;

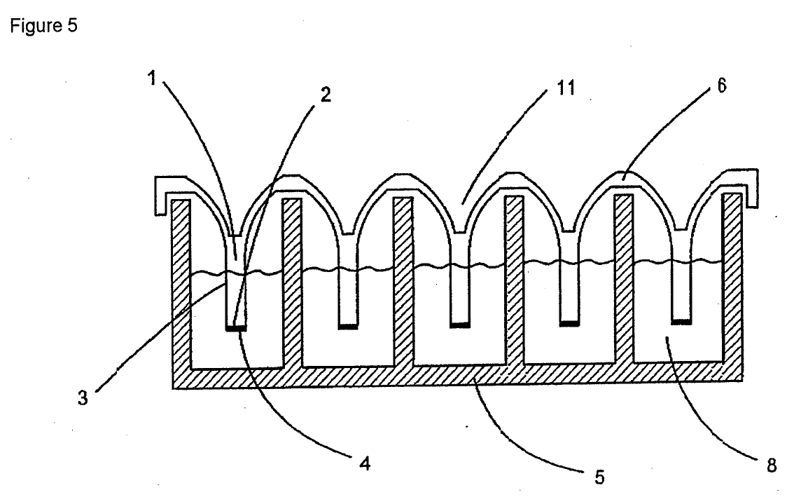

FIG. 5 shows a further embodiment of a lid element according to the invention;

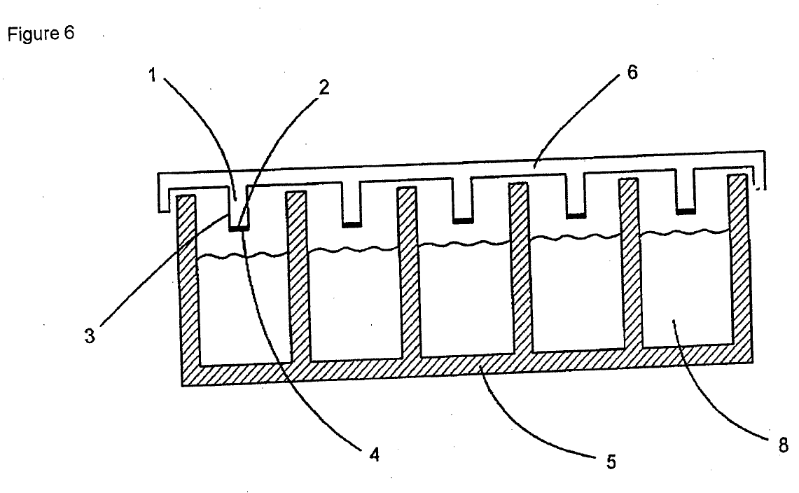

FIG. 6 shows a lid element according to the invention for determination of substance concentrations in a gaseous atmosphere above the liquid medium which contains the cells to be cultivated;

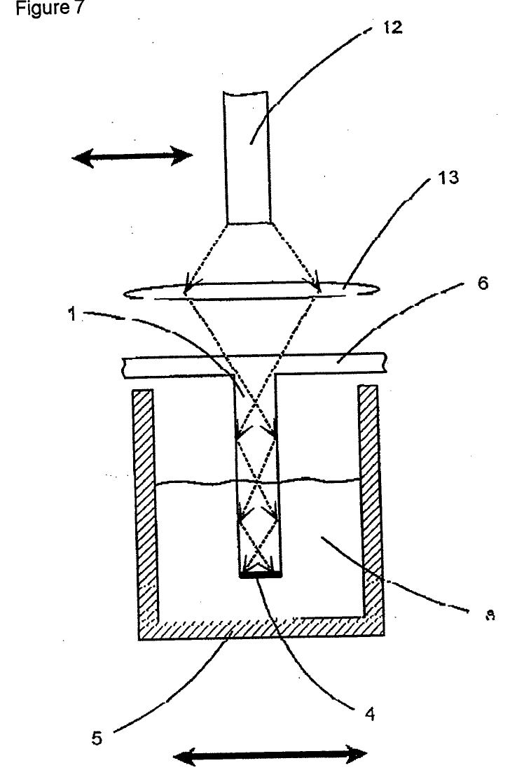

FIG. 7 shows, schematically, the illumination of an optically sensitive layer, which is arranged on an end surface of an optical waveguide which is in the form of a rod, within a liquid medium,

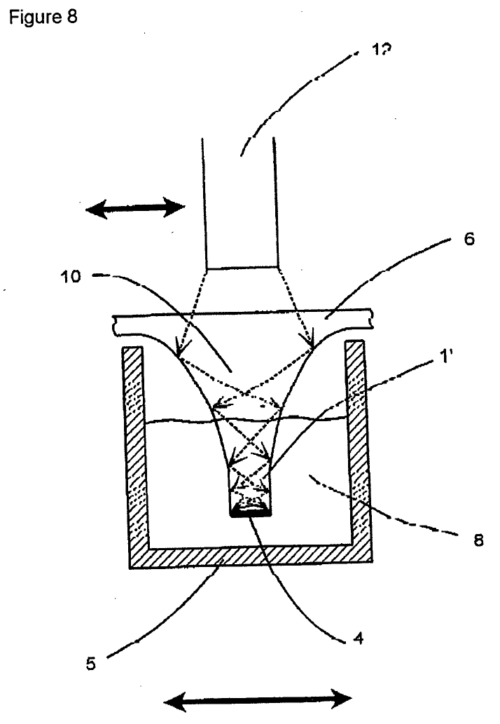

FIG. 8 shows, schematically, the illumination of an optically sensitive layer which is formed on an end surface of an optical waveguide with a funnel-shaped area;

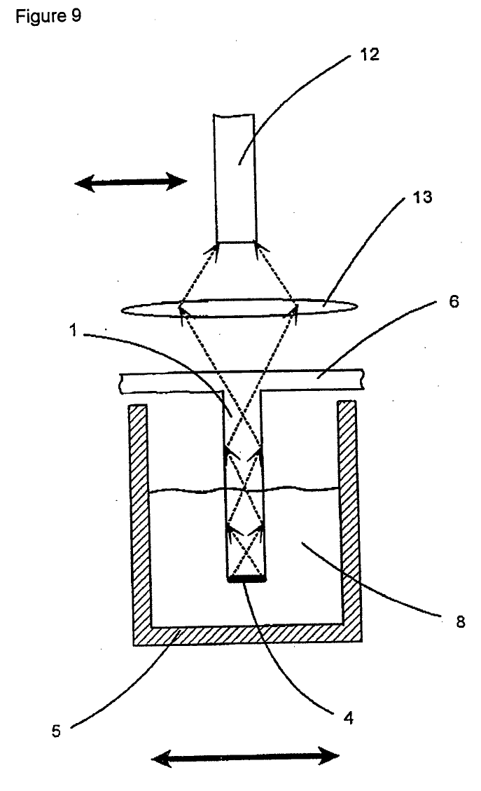

FIG. 9 shows, schematically, the light guidance of luminescence light, which is stimulated in an optically sensitive layer, from an optical waveguide which is in the form of a rod, and which luminescence light can be injected into an optical fiber and can be directed through this optical fiber onto an optical detector, which is not illustrated;

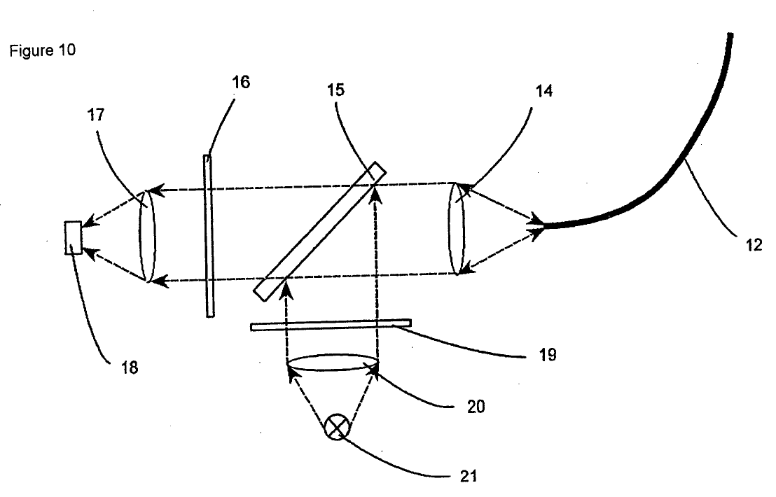

FIG. 10 shows, schematically, an optical layout for illumination of optically sensitive layers and for detection of light which is influenced by these layers, using an example with an optical fiber;

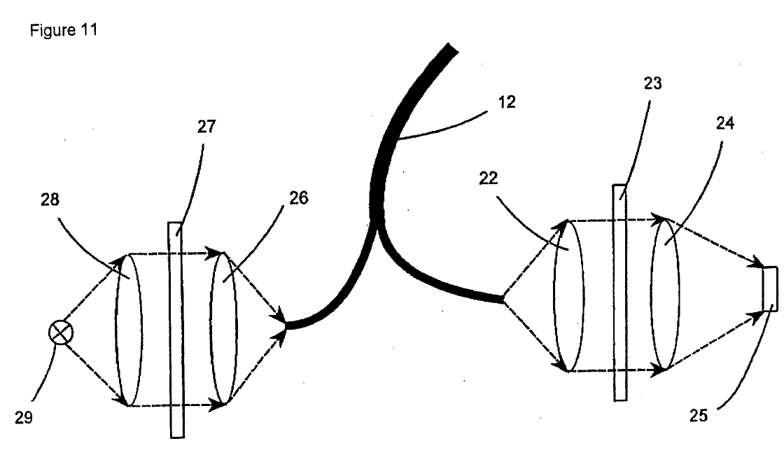

FIG. 11 shows a further example of an optical layout which is correspondingly suitable;

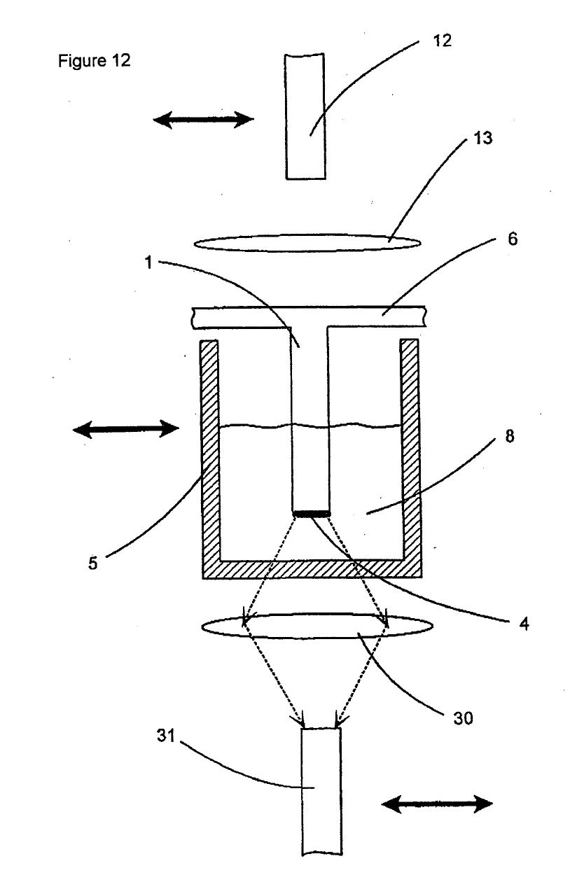

FIG. 12 shows, schematically, one option for arrangement of optical fibers via which the light from a light source, which is not illustrated, is directed onto an optically sensitive layer, and luminescence light which emerges from this layer is directed through the base of a cavity onto a detector, which is not illustrated;

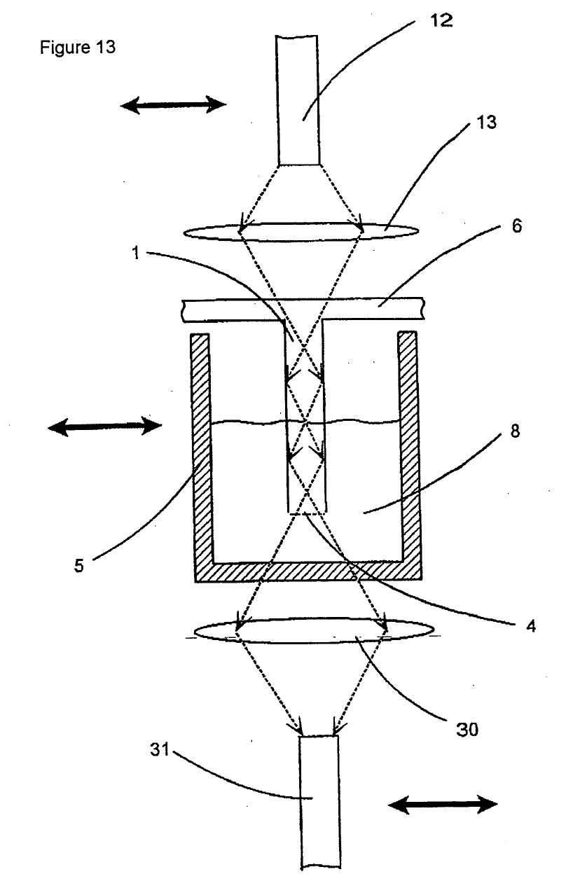

FIG. 13 shows, schematically, one option for arrangement of optical fibers via which the light from a light source which is not illustrated is directed onto an optically sensitive layer, and through this layer as well as the base of a cavity, onto a detector which is not illustrated;

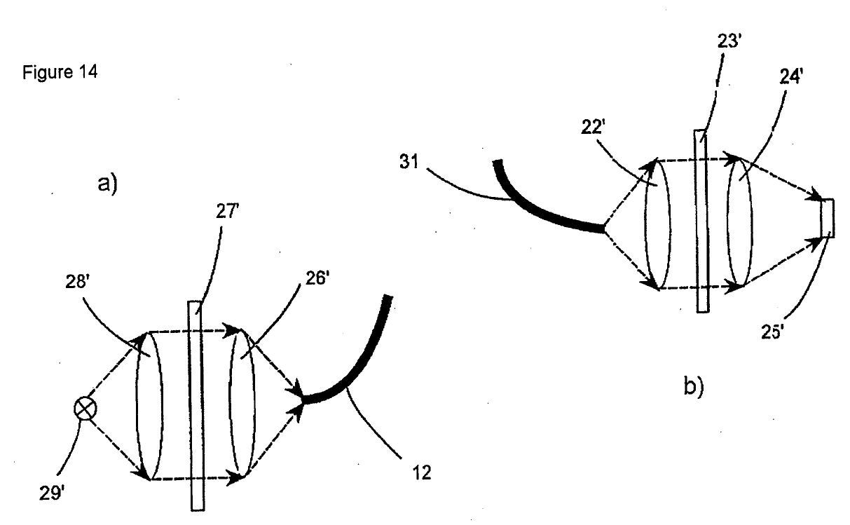

FIG. 14(a) shows an example of an optical layout as can be used for illumination of an optically sensitive layer, and

FIG. 14(b) shows an example of an optical layout for the detectors of the light from an optically sensitive layer, as can be used together with the examples illustrated in FIGS. 12 and 13;

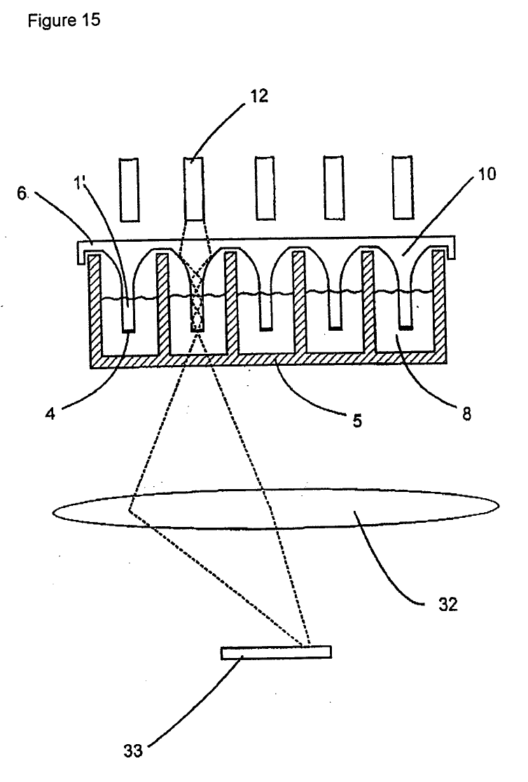

FIG. 15 shows, schematically, an example of an apparatus in which a measurement can be carried out simultaneously and with position resolution in two or more cavities in a cell culture vessel;

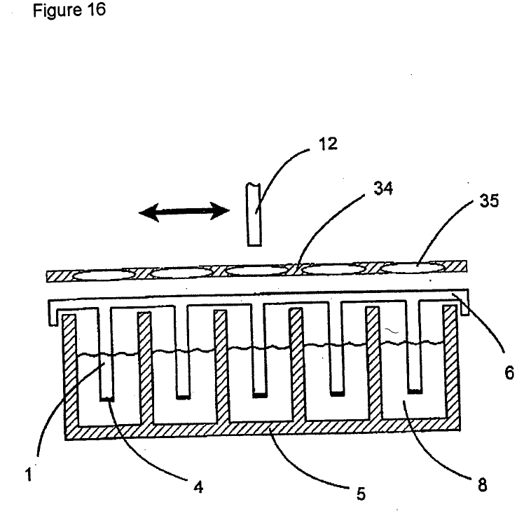

FIG. 16 shows an example of an apparatus with additional optical elements;

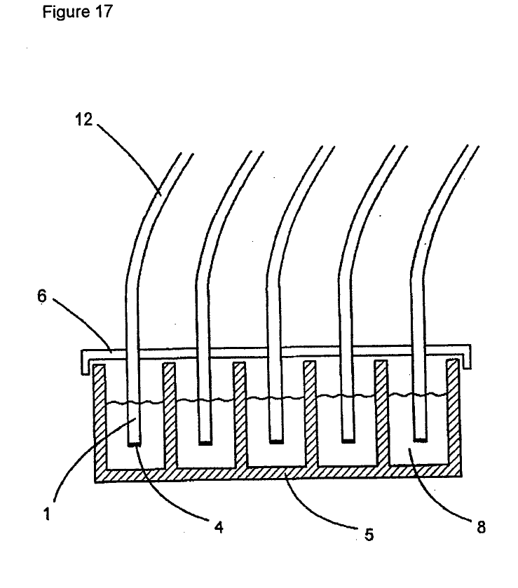

FIG. 17 shows an example with optical fibers as light-guiding elements;

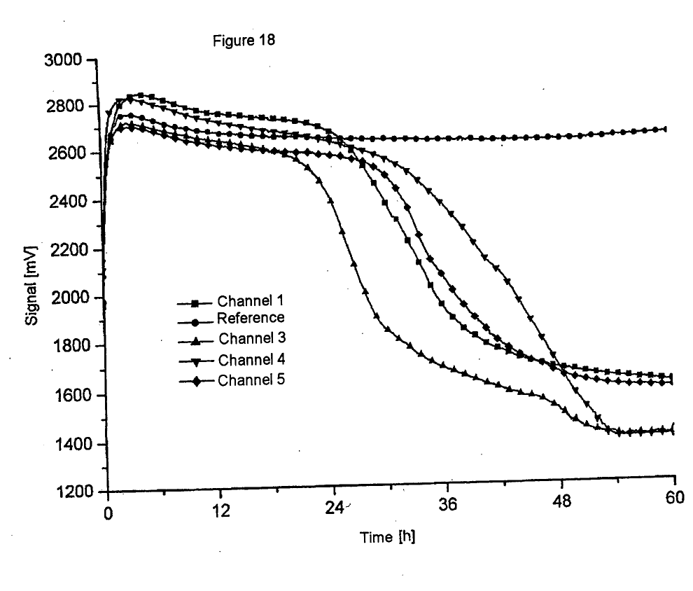

FIG. 18 shows a graph of measurement signal profiles which were measured in five cavities in a cell culture vessel, in uncorrected form and

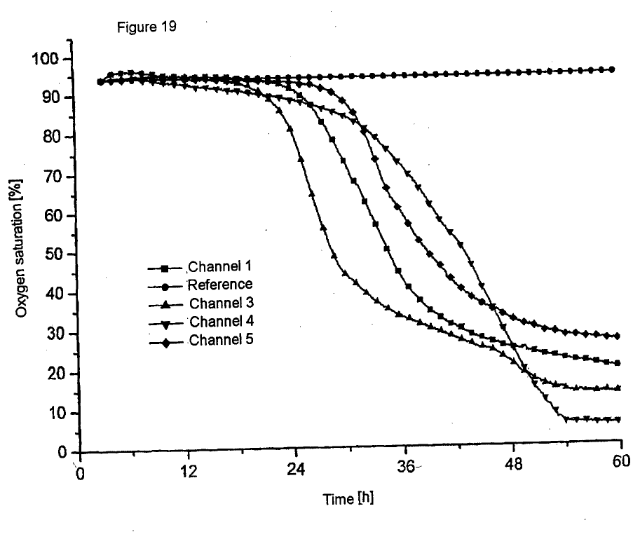

FIG. 19 shows a graph of the normalized measurement signal profiles as shown in FIG. 18.

An example of a lid element 6 according to the invention, as is placed on a cell culture vessel 5, having two or more cavities is shown, schematically, in FIG. 1.

In this case, an optical waveguide 1 which is in the form of a rod is provided for each cavity 8 on the lid element 6 according to the invention, with the entire lid element 6, including the optical waveguide 1 which is in the form of a rod, in this example having been produced from an optically transparent material. A lid element such as this may be produced, for example, using the injection-molding method from a suitable polymer plastic material which is transparent for light, such as PMMA.

The cavities 8 in the cell culture vessel 5 contain a liquid medium, as well as cells in this medium, as is indicated by the wavy line in the cavities 8.

In this example of a lid element 6 according to the invention, one optically sensitive layer 4 is formed on each of the lower end surfaces 2 of the optical waveguides 1, which are in the form of rods. However, optically sensitive layers 4 such as these may also be formed on their own or additionally on the outer envelope surface 3 of the optical waveguides 1, which are in the form of rods.

FIG. 2 shows the example shown in FIG. 1 in the form of a section plan view along the line A-A from FIG. 1. This clearly shows that the optical waveguides 1, which are in the form of rods, on the lid element 6 are in each case arranged centrally with respect to the individual cavities 8.

FIG. 3 illustrates a lid element 6 which has been modified from the example shown in FIG. 1. On its surface, this lid element 6 has a structure in the form of plano-convex lenses 9, which are arranged and formed with respect to in each case one optical waveguide 1, which is in the form of a rod.

In this case as well, this lid element 6 is in the form of a part and, in consequence, the plano-convex lenses 9 are also an integral component, of the lid element 6.

The example of lid elements 6 according to the invention as shown in FIG. 4 has optical waveguides 1 which have a funnel-shaped area 10, which merges into an area 1′ in the form of a rod.

In the example of a lid element 6 according to the invention as illustrated in FIG. 5, this lid element 6 has a structure in which concave depressions 11 are formed with respect to the individual cavities 8 and the optical waveguides 1, which are in the form of rods. Within these concave depressions 11, planar surfaces for light injection and/or outputting into and out of the optical waveguides 1, which are in the form of rods, are formed vis-à-vis the end surfaces 2 on which optically sensitive layers 4 are also formed in this example.

In the case of the lid element 6 according to the invention as shown in FIG. 6, the optical waveguides 1 which are in the form of rods are designed to be considerably shorter than the illustrated optical waveguides 1 which have been described in the previous examples and are in the form of rods, so that, here too, the optically sensitive layers 4 which are formed on the end surfaces 2 which point downwards are arranged above the liquid medium, within the cavities 8, in order to determine changing substance concentrations in a gaseous atmosphere.

However, as has already been mentioned in the general part of the description, this effect can also be achieved by appropriate spacers, which are formed on a lid element 6, or which can additionally be inserted between the lid element 6 and the cell culture vessel 5.

FIG. 7 shows an example of one possible way to guide the light for illumination of an optically sensitive layer 4, illustrated schematically. In this case, light from a light source which is not illustrated is directed via an optical fiber 12 onto a biconvex optical lens 13, and is passed by means of this optical lens 13 into the waveguide 1, which is in the form of a rod, of a lid element 6 which is illustrated in the form of an indication.

In this case, the optical lens 13 and the optical fiber 12 are chosen, and the element 1 which is in the form of a rod is of such a size, that the light is guided within optical waveguide 1, which is in the form of a rod, onto the optically sensitive layer 4, while maintaining total internal reflection conditions.

In a very largely analogous form, FIG. 8 once again illustrates an optical fiber 12, but in this case with a somewhat larger diameter, in which the light which emerges from an end surface is directed directly onto a planar surface of a lid element 6, and is directed through an optical waveguide 1 with a funnel-shaped area 10 and an area 1′ which is in the form of a rod, onto a sensitive layer 4 which is formed on the lower end surface 2 here, likewise maintaining total internal reflection on the outer envelope surfaces.

FIG. 9 is intended to indicate how luminescence light is directed from the optically sensitive layer 4, which then has appropriate characteristics, once again through the optical waveguide 1, which is in the form of a rod, via the biconvex optical lens 13 onto the end surface of an optical fiber 12 while maintaining total internal reflection conditions, for injection into the optical fiber 12. The luminescence light is passed via this optical fiber 12 onto an optical detector which is not illustrated.

FIG. 10 shows an optical layout as can be used in conjunction with the examples shown in FIGS. 7 to 9.

In this case, light from a light source 21 is directed through a biconvex optical lens 20, an optical filter 19 (which passes only light in the wavelength range which is suitable to stimulate luminescence) onto a dichroitic mirror 15, and from there via a further biconvex optical lens 14 and by means of this optical lens 14 onto an end surface of the optical fiber 12. This light is then passed through the optical fiber 12 into an optical waveguide 1, which is in the form of a rod (not illustrated here).

The luminescence light which is stimulated in the optically sensitive layer (which is not illustrated) can then be passed in the opposite direction through the optical fiber 12, and can be directed via the optical lens 14, through the dichroitic mirror 15, the optical filter 16 and via a further biconvax optical lens 17 onto an optical detector 18. In this case, the optical filter 16 blocks external light and stray light which is not in the same wavelength range as the luminescence light.

FIG. 11 shows a further example of an optical layout as can be used for an apparatus with a lid element 6 according to the invention. In this case, an optical fiber 12 is used which is divided into two parts. Instead of an optical fiber, it is also possible to use an optical fiber bundle, which is subdivided into two individual bundles. The part of an optical layout as illustrated on the left in FIG. 11 once again uses a light source 29, by means of which light is injected through two biconvex optical lenses 28 and 26, between which an optical filter 27 is arranged, into part of the optical fiber 12, and is directed via the optical fiber 12 and through an optical waveguide 1, which is not illustrated here but is in the form of a rod, onto an optically sensitive layer 4, which is likewise not illustrated.

Luminescence light and/or stray light then passes from the optically sensitive layer 4, after appropriate injection into the optical fiber 12, likewise via two biconvex optical lenses 22 and 24, between which an optical filter 23 is once again arranged, onto an optical detector 25.

In this case, the optical filters 27 and 23, in particular, are chosen such that the optical filter 27 transmits only light in the wavelength range which is required for stimulation of luminescence light and/or light scattering, and the optical filter 23 is permeable only for light in the wavelength range of the respective luminescence and/or scattered light.

Instead of the split optical fiber 12, as illustrated here, it is, however, also possible to use, in an analogous form, two individual optical fibers, which are connected to one another via a Y coupler.

FIG. 12 shows, schematically, one example of light guidance of luminescence light, in conjunction with an optically sensitive layer 4 whose luminescence can be varied as a function of the respective substance concentration within the liquid medium.

In this case, light from a light source which is not illustrated is once again injected via an optical fiber 12 and a biconvex optical lens 13 into an optical waveguide 1, which is in the form of a rod, in a lid element 6 according to the invention, and directed onto the optically sensitive layer 4 that is formed on the end surface 2 of the optical waveguide 1, which is in the form of a rod. The luminescence light which is produced in the optically sensitive layer is injected downwards through the base of the cavity 8 and via the biconvex optical lens 30 into a further optical fiber 31, from where it is directed onto an optical detector, which is not illustrated here.

FIG. 13 shows, schematically, one example of light guidance in conjunction with an optically sensitive layer 4 whose light transmission, absorption and/or light scatter can be varied as a function of the respective substance concentration within the liquid medium.

In this case, light from a light source which is not illustrated is once again injected via an optical fiber 12 and biconvex optical lens 13 into an optical waveguide 1, which is in the form of a rod, in a lid element 6 according to the invention, and is directed onto the optically sensitive layer 4 which is formed on the end surface 2 of the optical waveguide 1, which is in the form of a rod. In this case, a certain proportion of the light is absorbed or scattered by the optically sensitive layer 4 as a function of the respective substance concentration, so that only a position of the light can pass through the optically sensitive layer 4 and can be injected via the biconvex optical lens 30 into a further optical fiber 31, from where it can be directed onto an optical detector, which is not illustrated here.

FIGS. 7, 8, 9, 12, 13 and 16 indicate possible movements for positioning of the various elements, by means of double-headed arrows. It is possible for the cell culture vessel 5 to move with the lid element 6, with the optical components 12, 13, 30 and 31 being stationary, or for the cell culture vessel 5 with the lid element 6 to be stationary, and for the optical components 12, 13, 30 and 31 to move in synchronism with one another. In the stated cases, combined movement on different axes is also possible.

FIG. 14 shows optical fittings which can be connected to the optical fiber 12 and the optical fiber 31, corresponding to the examples shown in FIGS. 12 and 13.

In this case as well, there is once again a light source 29′, whose light is injected via biconvex optical lenses 28′ and 26′, between which an optical filter 27′ is once again arranged, from where it is directed onto the lid element 6 according to the invention, in order to illuminate the sensitive layer 4. The light which is transmitted through the optically sensitive layer 4, or the luminescence light which is stimulated in the optically sensitive layer 4, is injected into the optical fiber 31 and, after being output from this optical fiber 31, is likewise directed via two biconvex optical lenses 22′ and 24′ onto the detector 25′. In this case as well, an optical filter 23′ is arranged between the biconvex optical lenses 22′ and 24′ here.

FIG. 15 shows an example of one option by means of which measurements can be carried out in two or more cavities 8 in a cell culture vessel 5 at the same time by means of a lid element 6 according to the invention.

In this case, two or more optical fibers 12 are arranged above the lid element 6, and are positioned with respect to the optical waveguides 1, which are in the form of rods and project into the cavities 8.

The lid element 6 in this case has optical waveguides 1, which have funnel-shaped areas 10 and merge into an area 1′ which is in the form of a rod.

The light which emerges from the optical fibers 12 is directed through the optical waveguide 1 and through the optically sensitive layers 4, through the bases of the cavities 8 of the cell culture vessel 5 and via a biconvex optical lens 32 onto an optical detector 33.

In this case, the biconvex optical lens 32 is designed such that the light which emerges through the optically sensitive layers 4 from the optical waveguides 1, which are in the form of rods, is in each case directed from the individual cavities onto a specific surface area of the optical detector 33, which is in the form of a photosensitive array, so that simultaneous evaluation can be carried out for each individual cavity 8. The biconvex lens 32 may also advantageously be in the form of a lens system, in order to achieve optimum optical imaging characteristics. In this case, CCD arrays are particularly suitable for use as a photosensitive array.

FIG. 16 shows an example of an apparatus which uses a lid element 6 as shown in FIG. 1. Lid elements 6 according to the other examples which have been explained may also, of course, be used in a similar form.

In this case, a mount element 34 is arranged above the lid element 6, between an optical fiber 12 and a light source which is not illustrated.

In this example, biconvex optical lenses 35 are held fixed in the mount element 34 as beam-forming optical elements with respect to in each case one optical waveguide 1, which is in the form of a rod, so that the light which is output from the optical fiber 12 by means of relative movement (as is indicated by the double arrow and can be positioned with respect to the optical waveguides 1 which are in the form of rods,) and/or the light which is injected into the optical fiber 12 once again, can be focused in an advantageous manner by means of the biconvex optical lenses 35.

In this example, it is possible, by means of a specific arrangement of the mount element 34, to take account of different arrangements and, in particular, distances to a light source or, as shown here, to the end surface of the optical fiber 12 for injection and/or outputting of light with respect to the injection surface on the lid element 6 for the individual optical waveguides 1 which are in the form of rods. It is thus possible, for example, to move the mount element 34 in the vertical direction, that is to say upwards and downwards, and to fix it in an optimum position.

FIG. 17 shows an example of a lid element 6 with optical fibers 12 which merge within the cavities 8 into light-guiding elements, as optical waveguides in the form of rods.

The optical fibers 12 can be passed through corresponding apertures in the lid element 6, and can project into the interior of cavities 8 in a cell culture vessel 5. In this case, in the example illustrated here, those end surfaces of the optical fibers 12 which project into the interior of the cavities are provided with an optically sensitive layer 4.

The individual optical fibers 12 should be fixed to the lid element 6 such that the same lengths of each of them project into the interior of the cavities 8, so that measurements can in each case be carried out at the same distances from. the base, in the cavities 8 which are filled with the same volumes of the liquid medium.

FIG. 18 shows the experimentally determined measurement signal profile for an optical oxygen measurement in five cavities in a cell culture vessel 5 with 96 cavities (96 well microtitre plates). The optically sensitive layers are located on the end surfaces of the optical fibers 12, as is illustrated in FIG. 17. A layer as described in DE 198 31 770 A1 was used as the optically sensitive layer 4. The phase shift between the sinusoidal stimulation light and the sinusoidal luminescence light was measured as a measure of the oxygen concentration, using an optical layout as illustrated in FIG. 10. The optically sensitive layer for oxygen concentration determination was located 1.5 mm above the base of the cavities 8. Approximately 2*10⁴ cells of the cell number HL60 in the cavities numbers 1, 3, 4 and 5, which were respectively recorded by means of the measurement channel numbers 1, 3, 4 and 5. Cavity number 2, which was recorded by the measurement channel number 2, was not filled with cells. All the cavities were filled with 250 μl of cell culture medium (90% DMEM and 10% FCS deactivated). The cell culture vessel was located during the measurement in a breeding chamber at 37° C., with 100% relative humidity and at normal atmospheric pressure.

The measurement signal profiles over time as shown in FIG. 18 clearly show that a certain transient phase must be expected at the start of the measurements, in which precise evaluation is not possible. This is a result, in particular, of the required temperature equalization between the breeding chamber and the cell culture medium, since the cavities 8 were filled with cells and cell culture medium at room temperature, outside the breeding chamber. Once this time period which is required for this transient phase has elapsed, and which normally extends over a time of about 40 to 90 minutes, the measured measurement signals can be used.

The measurement signal profiles shown in FIG. 18 for the total of five measurement channels for the respective cavities 8 clearly show that maximum values were in each case measured at virtually the same time. Particularly for the reference channel, in this case the measurement channel number 2 which records the oxygen concentration in the cavity number 2, this results in a so-called reference value for the breeding chamber RWBₓ value, as the maximum signal in mV at a temperature of 37° C., with 100% relative humidity and at normal pressure, which takes account of the composition of the gas atmosphere within a breeding chamber and, in particular, of its oxygen concentration. Once this maximum value has been reached, FIG. 18 clearly shows that all of the measurement signals, including the measurement signal for the reference channel, fall.

The measurement values are reduced considerably after a measurement time of about 24 hours as a consequence of the metabolism-active cells which are contained in the cavity numbers 1, 3, 4 and 5 and which correspond to the measurement signals for the measurement channel numbers 1, 3, 4 and 5.

In order to improve the comparability and reproducibility, and to reduce measurement errors, it is possible in a simple form to normalize the measurement signals, and correspondingly normalized measurement signal profiles can be obtained from the graph in FIG. 19.

A normalization process such as this took account of the time at which the maximum value of the transient phase was reached in the cavity number 2, in which there were no cells, recorded by the measurement channel number 2 in FIG. 18, and referred to as the reference.

At this time, the respective difference between the measurement values from the individual measurement channel numbers 1, 3, 4 and 5 with respect to the RWB, value for the reference channel number 2 was determined as a value that was constant for each measurement channel. Taking this constant value and its mathematical sign into account, all of the measurement signals which were recorded over the time period were corrected for the respective measurement channel, so that all of the signal profiles for the RWB_{x value} have the same start point and, following this, those measurement signals which were recorded at later times were corrected by this constant value, with the measurement signal profiles effectively being shifted corresponding to this constant value, and taking account of its mathematical sign.

Furthermore, the measurement signal values from the individual measurement channel numbers 1, 3, 4 and 5 were corrected by means of values which vary with time. In this case, the individual measurement signal values from the individual measurement channel numbers 1, 3, 4 and 5 which were measured at different times were corrected by means of the value of the difference between the RWB, value and the measurement signal value for the reference channel number 2, as measured at this time.

As is also evident from the graph shown in FIG. 19, normalization, was also carried out with respect to the actually measured oxygen concentration, taking into account the oxygen concentration within the gas atmosphere in the breeding chamber in which the measurements were carried out, and taking account of the environmental air atmosphere, at the same temperature and with the same air humidity.

1. A lid element which can be fitted to cell culture vessels having at least one cavity, with light-guiding elements being provided on the lid element;
in each case at least one light-guiding element projecting into the interior of a cavity in the cell culture vessel on the lid element is fitted to the cell culture vessel; with a liquid medium being contained in cavities, and cells as well being contained in at least one cavity;
and in each case at least one optically sensitive layer which is suitable for detection of chemical substance concentrations which vary within the cavities being formed on one end surface and/or on the 20 outer envelope surface of the light-guiding element.

2. The lid element as claimed in claim 1, characterized in that the optically sensitive layer changes its optical characteristics with respect to the luminescence intensity and/or decay time, light transmission or light scatter, as a function of the respective changing chemical substance concentration in the cavity.

3. The lid element as claimed in claim 1, characterized in that the light-guiding elements are optical waveguides in the form of rods.

4. The lid element as claimed in claim 3, characterized in that the surface of the lid element forms a structure in the area of the light-guiding elements.

5. The lid element as claimed in claim 4, characterized in that the structure is in the form of convex projections or concave depressions.

6. The lid element as claimed in claim 5, characterized in that the convex projections form planar convex optical lenses, or the concave depressions form concave optical lenses.

7. The lid element as claimed in claim 5, characterized in that a structure which is in the form of depressions is funnel-shaped, and a planar surface is formed within the funnel-shaped area for injection and/or outputting of light to or from the light-guiding elements.

8. The lid element as claimed in claim 1, characterized in that the light-guiding elements have an area which is in the form of a funnel, a truncated cone or a truncated pyramid, and merges into an area which is in the form of a rod.

9. The lid element as claimed in claim 1, characterized in that the optically sensitive layer is composed of a substance which is suitable for luminescence stimulation, or contains such a substance.

10. The lid element as claimed in claim 1, characterized in that the light-guiding elements have a circular, oval, triangular or polygonal cross section.

11. The lid element as claimed in claim 1, characterized in that spacers or openings are provided on the lid element.

12. The lid element as claimed in claim 11, characterized in that the openings are closed by gas-permeable membranes.

13. The lid element as claimed in claim 1, characterized in that the surface of the lid element is provided with a layer which reflects or absorbs light, except for areas for the injection and/or outputting of light into/from the light-guiding element or elements.

14. An apparatus having a lid element as claimed in claim 1, for optical determination of the metabolism activity of cells which are contained in a liquid medium in cavities in cell culture vessels, characterized in that light from at lease one light source is directed through light-guiding elements, which are provided on the lid element onto or through optically sensitive layers which are in the form of light-guiding elements, and at least one optical detector is provided for measurement of stimulated luminescence light in the optically sensitive layer and/or light transmitted through the optically sensitive layer, and/or light scattered through the optically sensitive layer.

15. The apparatus as claimed in claim 14, characterized in that the light is guided to and/or from the optically sensitive layer via at least one optical fiber.

16. The apparatus as claimed in claim 14, characterized in that the cell culture vessel and light source or end surfaces of the optical fibers can be moved relative to one another in order to output and/or inject light, and can be positioned on the cell culture vessel with respect to a light-guiding element in the lid element.

17. The apparatus as claimed in claim 16, characterized in that at least one optical detector can additionally be moved and positioned on the cell culture vessel relative to the light-guiding elements in the lid element.

18. The apparatus as claimed in claim 14, characterized in that the light source or the end surface for outputting light from optical fibers and/or from the at least one optical detector is arranged above the lid element and openings of the cavities in the cell culture vessel.

19. The apparatus as claimed in claim 14, characterized in that the at least one optical detector is arranged underneath the base of cavities in a cell culture vessel.

20. The apparatus as claimed in claim 14, characterized in that the cell culture vessel is a microtitre plate.

21. A method for optical determination of metabolism activities of cells using an apparatus as claimed in claim 14, characterized in that at least one optical detector is used to detect at least one substance concentration, which changes as a consequence of metabolism activity in the cells, within cavities in a cell culture vessel using optical characteristics of optically sensitive layers which change as a function of the changing substance concentration.

22. The method as claimed in claim 21, characterized in that the intensity of the light which strikes the at least one optical detector is measured.

23. The method as claimed in claim 21, characterized in that the intensity of luminescence light which is stimulated in the optically sensitive layer is detected.

24. The method as claimed in claim 21, characterized in that the time decay response or the phase shift of luminescence light which is stimulated in the optically sensitive layer is determined.

25. The method as claimed in claim 21, characterized in that the intensity of light which is transmitted through and/or scattered by the optically sensitive layer is measured.

26. The method as claimed in claim 21, characterized in that the measurements are carried out repeatedly at time intervals which can be predetermined for in each case one cavity.

27. The method as claimed in claim 21, characterized in that the concentration and/or the change in the concentration of O₂, CO₂, H⁺, H₂, H₂S, NH⁴⁺ and/or the pH value are/is determined.

28. The method as claimed in claim 21, characterized in that the concentration and/or the change in the concentration of enzyme substrates, produced by the metabolism activity of the cells is determined by enzyme sensors as the optically sensitive layer.

29. The method as claimed in claim 38, characterized in that glucose and/or lactate are/is determined by means of enzyme sensors as the optically sensitive layer.

30. The method as claimed in claim 21, characterized in that at least one cavity in a cell culture vessel with a lid element is not filled with cells, and is used as a reference for substance concentration determination and for its change, by means of optically sensitive layers.

31. The method as claimed in claim 21, characterized in that the change within the cavities above the liquid medium is determined.
